(19) United States
(12) Patent Application Publication
(10) Pub. No.: US 2003/0212147 A1
(43) Pub. Date: Nov. 13, 2003

(54) Enzyme activated chemotherapy of liver cancer

(76) Inventor: Sum Man Stephen Chung, Hong Kong (CN)

Correspondence Address: PENNIE AND EDMONDS, 1155 AVENUE OF THE AMERICAS, NEW YORK, NY, 100362711

(21) Appl. No.: 10/332,246

(22) PCT Filed: Jul. 12, 2001
(86) PCT No.: PCT/US01/21975

(51) Int. Cl. G01N 33/574; C12Q 1/26; A61K 31/05

(52) U.S. Cl. 514/732; 435/7.23; 435/25

(57) ABSTRACT

The present invention provides methods of screening for anti-cancer drugs based on the activities of overexpressed enzymes by the cancer cells to convert non-toxic compounds to cytotoxic drugs which can specifically kill these cancer cells. In particular, the present invention provides methods of screening for anti-cancer drugs which can be used to treat liver cancer, especially hepatocellular carcinoma. The present invention also provides methods of treating cancers, in particular liver cancer, especially hepatocellular carcinoma, by administering to a subject the drugs selected by the screning methods of the present invention. In a particular embodiment, naphthalene or a derivative thereof is administered to a subject. The present invention further provides pharmaceutical compositions for treating or ameliorating cancers as well as kits containing such compositions.

### 1. INTRODUCTION

The present invention relates to methods of screening for anti-cancer drugs based on the activities of overexpressed enzymes by the cancer cells to convert non-toxic chemicals or compounds to cytotoxic drugs which can specifically kill these cancer cells. In particular, the present invention relates to methods of screening for anti-cancer drugs which can be used to treat liver cancer, especially hepatocellular carcinoma (HCC). The present invention also relates to methods of treating or ameliorating cancers, in particular, liver cancer, especially hepatocellular carcinoma, by administering the drugs selected by the screening methods of the present invention. The present invention further relates to pharmaceutical compositions for treating or ameliorating cancers, as well as kits containing such compositions.

### 2. BACKGROUND

Liver cancer, especially hepatocellular carcinoma (HCC), is one of the leading causes of cancer related death in Hong Kong and China. It is also a significant health problem in many other countries, including the United States and European countries. Currently the only effective treatment is by surgical removal of the cancerous tissues, if the cancer is discovered at early stages, its size remains small, and is not metastasized to other sites. However, most cases of HCC are asymptomatic at early stages and, when the patients are diagnosed as having HCC, it is usually too late for surgical intervention. Various chemotherapy regimen, including hepatic intraarterial chemotherapy (HIA) and chemoembolization using conventional chemotherapeutic agents, have shown limited success (Venook, A. P., 2000, Regional strategies for managing hepatocellular carcinoma. Oncology (Huntingt) 14:347-54).

Aldose reductase (AR) is a nicotinamide adenine dinucleotide phosphate (NADPH)-dependent enzyme that was originally identified by its ability to reduce glucose to sorbitol and is present in kidney and testes as well as in many other tissues. AR is also efficient in reducing various aromatic and aliphatic aldehydes, e.g., glyceraldehyde, benzaldehyde and pyridine aldehyde (Inazu, N., et al., 1994, J. Biochem. (Tokyo) 115:991-999) and it has been suggested that AR may be responsible for detoxifying toxic lipid aldehydes produced during oxidative stress and other harmful aldehydes produced as a result of cellular metabolism (V. Jagt et al., 1992, J. Biol. Chem., 267:4364-4369; V. Jagt et al., 1995, Biochem. Biophs. Acta 35 1249:117-126). On the other hand, it was also reported that AR is overexpressed in stomach carcinoma cell lines (Ax, et al., 2000, Biochem. Pharmacol. 59:293-300) and colon-cancer cell lines (Akashi, et al., 2000, Int. J. Cancer 88:873-80). In addition, it was previously shown that about 29% of HCC overexpressed AR, and about 54% overexpressed ARL-1, an aldose reductase-like protein that is 71% identical to AR in amino acid sequence, with similar enzymatic activity as AR (Cao, D. L. Fan, S. T., and Chung, S. S. M., 1998, Identification and characterization of a novel human aldose reductase-like gene. J. Biol. Clem. 273a:11429-35). As described above, AR and ARL-1 have broad substrate specificity and these two enzymes can reduce a broad range of aliphatic and aromatic aldehydes. Accordingly, they are generally considered to be enzymes that detoxify anti-cancer drugs (Hyndman, et al., 1999, Enzymol. Molec. Biol. Carbonyl Metab 7:427-434, Weiner et al. ed., Kluwer Academic/Plenum Pub. New York). Furthermore, there is evidence that these enzymes can indeed make cancer cells more resistant to some anti-cancer drugs (Lee, et al., 2001, Anti-cancer Drugs 12:129-132). However, the potential of these enzymes to be utilized to convert non-toxic chemicals to cytotoxic drugs so as to specifically kill cancer cells that overexpress the enzymes, has not been actively explored.

In an unrelated project to understand the mechanism of naphthalene-induced cataract, Lee et al. found that AR is responsible for converting the relatively non-toxic naphthalene-1,2-dihydrodiol (ND) to 1,2-dihydroxynaphthalene, which then auto-oxidizes itself to form the 1,2-naphthoquinone (NQ) that is highly cytotoxic (Lee, A. Y. W. and Chung, S. S. M., 1998, Involvement of aldose reductase in naphthalene cataract. Invest. Ophthalmol. Vis. Sci. 39:193-97, which is hereby incorporated by reference in its entirety). The proposed mechanism of naphthalene cataract is shown in FIG. 1 (Lee, A. Y. W. et al., 1998, supra; van Heyningen, R., 1967, The metabolism of naphthalene and its toxic effect on the eye. Biochem. J. 102:842-852).

### 3. SUMMARY OF INVENTION

The present invention is based, in part, on the observation by the present inventor that a large percentage of HCC overexpresses either AR or the related enzyme ARL-1, and that AR and ARL1-1 can convert non-toxic ND to highly cytotoxic NQ (see FIG. 1). Accordingly, naphthalene or a non-toxic derivative thereof can be used as a drug to specifically kill liver cancer cells that overexpress the two genes encoding AR and ARL-1, respectively. Thus, the present invention provides methods of screening for anti-cancer drugs which are the substrates for the enzymes overexpressed by certain cancer cells and are converted to cytotoxic drugs by the activities of the enzymes so as to specifically kill the cancer cells by the virtue of overproduction of these enzymes by the cancer cells. The present invention provides a simple, efficient, and most importantly target specific methods for screening anti-cancer drugs. Namely, the present invention provides the methods of screening for an anti-cancer drug comprising: contacting a cancer cell overexpressing an enzyme with a candidate drug in the presence or absence of an inhibitor to the enzyme; measuring a level of a substance correlatable with the level of cell death as a result of exposure of the cell to the drug; and comparing the levels of the substance in the presence and absence of the inhibitor, wherein a difference in the level of the substance in the absence of the inhibitor indicates that the drug has been converted by the enzyme to have an anti-cancer activity. In a specific embodiment, the present invention provides methods of screening for the anti-cancer drugs to treat hepatocellular carcinoma that overexpresses AR and/or ARL-1.

The present invention also provides methods of treating or ameliorating cancer comprising administering to a subject in need of a treatment a therapeutically effective amount of the drug having an anti-cancer activity as determined by the screening method of the present invention. In a specific embodiment, the cancer to be treated is hepatocellular carcinoma overexpressing AR and/or ARL-1. In another specific embodiment, the drug to treat hepatocellular carcinoma is naphthalene or a derivative thereof, e.g., naphthalene 1,2-oxide and naphthalene 1,2-dihydrodiol.

The present invention further encompasses pharmaceutical compositions for treating or ameliorating cancer comprising a therapeutically effective amount of a drug having an anti-cancer activity as determined by the screening method according to the present invention. In a specific embodiment, the cancer to be treated is hepatocellular carcinoma overexpressing AR and/or ARL-1. In another specific embodiment, the drug having an anti-cancer activity is naphthalene or a derivative thereof. Furthermore, the present invention provides a kit comprising in one or more containers the pharmaceutical compositions of the present invention.

### 3.1 Definitions

The term “a drug having an anti-cancer activity” as used herein refers to a drug that by itself may have very low to undetectable levels of toxicity to either normal cells or cancer cells but is converted to be highly cytotoxic to the cancer cells by an enzyme overexpressed by the cancer cells so that the drug can halt the growth of the cancer cells and/or kill the cancer cells selectively with very little to no harm to normal cells.

The term “liver cancer” as used herein refers to cancers found in liver, which may originate from liver (as referred herein as “hepatocellular carcinoma” or “HCC”) or from other organs and have spread to the liver as a result of metastasis.

The term “therapeutically effective amount” as used herein refers to the amount of a drug or derivative thereof that reduces or ameliorates the severity, the duration and/or the symptoms associated with a cancer in a subject.

The term “pharmaceutically acceptable carrier” as used herein refers to an inert accessory substance that forms a vehicle for a drug and is approved by a regulatory agency of the Federal or a state government or listed in the U.S. Pharmacopeia or other generally recognized pharmacopeia for use in animals, and more particularly in humans.

### 4. BRIEF DESCRIPTION OF THE FIGURES

FIG. 1 shows the proposed mechanism for the biotransformation, involving aldose reductase (AR), of non-toxic naphthalene into highly cytotoxic 1,2-naphthoquinone.

FIG. 2 shows the Expression of AR gene in HepG2 cells at various time in hypertonic medium. The band at 2.37 kb represents an improperly processed AR mRNA and the band at 1.35 kb represents the functional AR mRNA. GADPH is a gene not induced by hypertonicity and shown to indicate the relative amount of mnRNA loaded onto each lane. Lane C represents an isotonic control.

FIG. 3 shows a graph indicating the cytotoxicity of naphthalene 1,2-dihydrodiol (ND) in HepG2 cells cultured in isotonic (I) or hypertonic medium (H) in the presence or absence of AR inhibitor (ARI), AL1576 (Alcon Laboratories, Fort Worth, Tex.). LDH released in the medium was assayed 5 hours after the addition of ND. The data are expressed as mean±S.D. (n=3) and the statistical analysis was performed by the one-way ANOVA (* indicates p<0.001).

### 5. DETAILED DESCRIPTION OF THE INVENTION

### 5.1 Screening Assays

It is well known that various cancer cells exhibit altered levels of gene expression compared to the normal cells. When the expressions of certain enzymes are increased in cancer cells, the catalytic activities of these enzymes can be effectively utilized to turn non-toxic compounds into highly cytotoxic compounds in a very selective manner using the screening methods of the present invention.

The present methods for screening potential anti-cancer drugs not only eliminate general chemotherapeutic agents which may be highly toxic to normal cells but selecting out effective anti-cancer agents that are specific to cancer cells by virtue of overexpression of certain enzymes by the cancer cells.

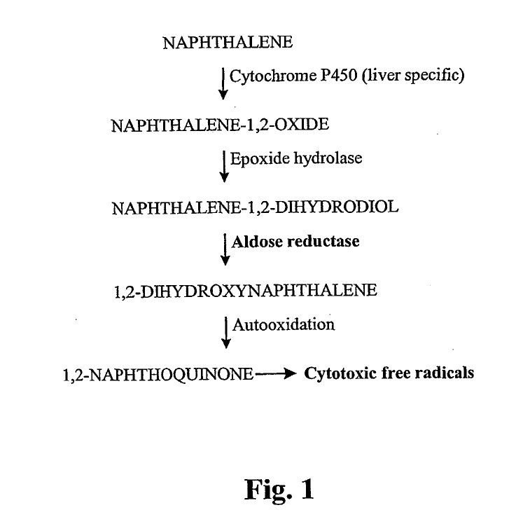

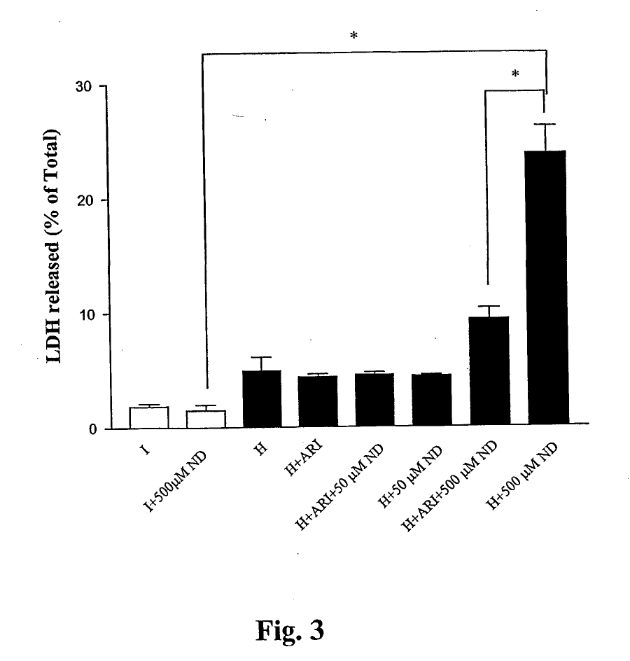

Using the screening methods of the present invention, naphthalene and some of its derivatives are found to have potent anti-cancer activity against the cancer cells, such as hepatocellular carcinoma, which overexpress AR and/or ARL-1 (see 7.2.2, infra; and FIG. 3). FIG. 1 shows the proposed mechanism for the biotransformation, involving aldose reductase (AR), of non-toxic naphthalene into highly cytotoxic 1,2-naphthoquinone (NQ).

As shown in FIG. 3, the toxic effect of ND converted to NQ is correlated with increased expression of AR by HepG2 cells as HepG2 cells cultured with 500 μM of ND in isotonic medium (control cells), which does not affect the expression of AR-by HepG2 cells, showed no sign of toxic effects, whereas those cultured with the same amount of ND in hypertonic medium, which increases the expression of AR, released more than 10 times higher LDH than that released by the control cells. Furthermore, the presence of AR inhibitor protected the HepG2 cells from the toxic effects by ND.

Accordingly, the present invention provides a method of screening for an anti-cancer drug comprising:

contacting a cancer cell overexpressing an enzyme with a candidate drug in the presence or absence of an inhibitor to the enzyme;

measuring a level of a substance correlatable with the level of cell death as a result of exposure of the cell to the drug; and

comparing the levels of the substance in the presence and absence of the inhibitor;

wherein a difference in the level of the substance in the absence of the inhibitor indicates that the drug has an anti-cancer activity against the cancer cell overexpressing the enzyme.

In a specific embodiment, the cancer is liver cancer, including those metastasized from other organs and those originating from the liver, preferably HCC, that overexpresses AR and/or ARL-1. The inhibitor can be any antagonist of AR and/or ARL-1, including AL1576 (2,7-difluorospirofluorene-9,5′-imidazolidine-2′4′-dione; Alcon Laboratories, Fort Worth, Tex.), Eparestat (E-3-carboxymethyl-5-[(2E-methyl)-3-phenylpropenylidene]-rhodanine; Ono Pharmaceutical Co. Ltd., Japan), Sorbinil (S-6-fluorspirochroman-4,5′imidazolidine-2′,4′-dione; Pfizer Inc., New York), Zopolrestat (3,4-dihydro-4-oxo-3-{[5-(tarifluoromethyl)-2-benzothiazoly]methyl}-1-phthalazineacetic acid; Pfizer), Tolrestat (N-[5-(trifluoromethyl)-6-methoxy-1-naphthalenyl]-N-methylglycine; Wyeth-Ayest Laboratories) and Fidarestat ((+)-(2S,4S)-6-fluoro-2′5′dioxospirochroman-4,4′-imidazolidine-2-carboxyamide; Sanwa, Japan).

Cytotoxicity of candidate drugs can be measured by various methods known to ordinary skill in the art, including, but not limited to, LDH-release assay and Cr⁵¹-release assay.

By employing various cancer cell cultures which overexpress particular enzymes having broad spectrum of substrates, the screening method of the present invention can be applied to drug discoveries for other types of cancers than liver cancer.

### 5.2 Therapeutic Utility of the Drugs Selected by the Screening Methods

It is often difficult to treat hepatocellular carcinoma (HCC) because of the variation in underlying etiology of the liver diseases, such as hepatitis B, hepatitis C, cirrhosis, etc., and the therapeutic efficacy of surgery and/or various types of chemotherapy are greatly affected by the balance between tumor growth and adequate hepatic reserve (A. Venook, 2000, Regional strategies for managing hepatocellular carcinoma, Oncology 14(3):347-354, which is hereby incorporated by reference in its entirety). Systemic chemotherapies are often inappropriate because of their systemic toxicities to even normal cells. Accordingly, regional hepatic drug delivery is often a preferred method for treating HCC. Examples of regional therapy include hepatic intraarterial chemotherapy (HIA), chemoembolization, and internal radiotherapy. However, general toxicity of chemotherapeutic drugs, such as floxuridine and mitomycin, and that of radiotherapy still pose a significant risk for hepatic failure to patients with inadequate hepatic reserve even by a regional therapeutic method.

The anti-cancer drugs selected by the method of present invention are much more specific to particular cancer cells and, therefore, safer and yet much more effective than the conventional chemotherapeutic agents especially in regional therapies. For example, the drugs may be delivered using an implanted pump into the hepatic artery (HIA method) of a subject with HCC. This may be especially effective since it was reported that liver tumors receive 80% of their blood supply from the hepatic artery, whereas normal liver tissue receives the most of its blood supply from the portal vein (Venook, 2000, supra; Breedis et al., 1954, The blood supply of neoplasms in the liver. Am. J. Pathol. 30:969-985). Alternatively, the drugs of the present invention may be administered in combination with other chemotherapeutic agents, such as floxuridine, leucovorin, adriamycin, Platinol (FLAP), 5-FU (PIAF) and mitomycin in HIA chemotherapy.

Chemoembolization is another method available for regional delivery of the drugs of the present invention. This method uses the combination of vascular occlusion and HIA chemotherapy. In this method, the occlusion of the hepatic artery causes the blockage of the arterial blood supply to the tumor and leads to tumor ischemia without affecting the other regions of the liver. In addition, the simultaneous addition of the drugs intraarterially effects the increased distribution and the dwell time of the drugs in the affected region (Venook, 2000, supra). As embolization materials, a gelatin sponge (Venook, et al., 1990 Chemoembolization for hepatocellular carcinoma. J. Clin. Oncol. 8(6): 1108-1114), collagen (Daniels et al., 1988, Collagen chemoembolization: Pharmacokinetics and tissue tolerance of cisplatin in liver and kidney. Cancer Res. 48:2446-2450), polyvinyl alcohol (Ajani et al., 1988, Islet cell tumors metastatic to the liver: Effective palliation by sequential hepatic artery embolization. Ann. Intern. Med. 108:340-344), and microspheres (Ho et al., 1997, Tumour-to-normal uptake ratio of 90-Y microspheres in hepatic cancer assessed with 99Tcm macroaggregated albumin. Br. J. Radiol. 70:823-828).

The drugs of the present invention can be also used in combination with Lipiodol. Lipiodol is known as a imaging contrast medium used for limphangiography. It is an ethyl ester of the fatty acid of poppyseed oil and contains 38% iodine by weight (Venook, 2000, supra). Since Lipiodol concentrates in neoplastic lesions in the liver when administered intra-hepatic-arterially and, therefore, enhances drug delivery. Accordingly, the drugs of the present invention may be dissolved into Lipiodol and administered HIA or with concomitant chemoembolization.

In addition, the drugs selected by the screening methods of the present invention may be administered systemically by oral administration (e.g., tablets and capsules) or parenteral administration (e.g., intravenous, intramuscular and subcutaneous injections) because of their reduced systemic toxicity.

These treatments may be employed especially for patients who are highly susceptible to systemic toxicities and/or have multifocal tumors which preclude surgical interventions. Furthermore, these treatments may be applied to patients who are waiting for the transplant in order to halt the tumor growth or dissemination of cancer cells to the other parts of the body.

Appropriate dosages of the drugs of the present invention may be determined by experiments in various animal models for specific types of cancer, for example, a rat hepatoma model using N-nitroso-N-methylurea (Diwan, B. A. et al., 1985, N-Nitroso-N-methylurea initiation in multiple tissues for organ-specific tumor promotion in rats by phenobarbital. J. Natl. Cancer Inst. 75:1099-1105) or 2-acetylaminofluorene (Hadjiolov, N. et al., 1995, Early initiating and promoting effects in 2-AAF-induced rat liver carcinogenesis: an immunohistochemical study. Cancer lett. 98:39-46). It is known that, similarly to human HCC, rat hepatomas also overexpress AR (Takahashi, M. et al., 1995, Elevation of aldose reductase gene expression in rat primary hepatoma and hepatoma cell lines: implication in detoxification of cytotoxic aldehydes. Int. J. Cancer 62(6):749-54) and aldose reductase-like protein ARL-1 (Zeindl-Eberhart, E. et al., 1997, Futher characterization of a rat hepatoma-derived aldose-reductase-like protein—organ distribution and modulation in vitro. Eur. J. Biochiem. 247:792-800). Furthermore, human HCC samples can be orthotopically implanted into nude mice, where they will develop into liver cancer (Sun, F. X. et al., 1996, Metastatic models of human liver cancer in nude mice orthotopically constructed by using histologically intact patient specimens. J. Cancer Res. Clin. Oncol. 122(7):397-402). The nude mouse model has the advantage of testing on actual human HCC.

In the rabbit cataract model, about 1 g/kg body weight of naphthalene develop cataract and degeneration of the retina (van Heyningen, R. et al., 1967, The metabolism of naphthalene and its toxic effect on the eye. Biochem. J. 102:842-852). Accordingly, this is most likely the toxic level for human as well for oral administration of naphthalene. Higher levels of drugs can be used if administered by chemoembolization, which delivers drugs locally to the lobe of the liver affected by cancer.

### 6. EXAMPLES

It is understood that modifications which do not substantially affect the essential activity of each embodiment of the present invention are also within the scope of the present invention. Accordingly, the following examples are intended to illustrate but not limit the present invention.

### 6.1 Materials and Methods

### 6.1.1 Chemicals

1,4-naphthoquinone (NQ) was purchased from Aldrich Chemical Co. (Milwaukee, Wis.); naphthalene-1,2-dihydrodiol (ND) from BioMol Research Laboratories (Plymouth, Pa.); Naphthalene and betaine from Sigma (St. Louis, Mo.); and aldose reductase inhibitor (ARI) AL1567 was a gift from Alcon Laboratories (Fort Worth, Tex.). MEM medium, Dulbecco's modified Eagle's medium with 4.5 g/L glucose, fetal bovine serum, penicillin, streptomycin, non-essential amino acids, glucose, and sodium pyruvate were obtained from Life Technologies (Gaithersburg, Md.). Cytotoxicity Detection Kits (LDH) were obtained from Roche Molecular Biochemicals (Mannheim, Germany).

### 6.1.2 Cell Cultures

HepG2 cells, a human liver cell line, were purchased from the American Type Culture Collection (Rockville, Md.). The basic (isotonic) medium was MEM medium supplemented with 10% fetal bovine serum, 100 U/ml penicillin, 100 μg/ml streptomycin, 0.1 mM MEM non-essential amino acids, and 1 mM sodium pyruvate. Medium was made hypertonic by adding NaCl to 100 mM, and the osmolarity was about 500 mosmol/kg as determined by osmometer. ND was dissolved in 20% ethanol to make a 50 mM stock solution. NQ was dissolved in 100% ethanol to make a 50 mM stock solution. AL1576 was dissolved in 20 mM NaOH to make a 20 mM stock solution.

Cells were first cultured in isotonic medium until 70% confluent. The medium was then made hypertonic by NaCl and incubated for 72 hours. The cells were then seeded onto 24-well plate at 8×10⁴ cells/well and incubated for 24 hours in hypertonic medium. Cells for isotonic control were processed in parallel except that no NaCl was added. ND and AR inhibitor AL1576 were added as indicated.

### 6.1.3 LDH Assays

Cytotoxicity was measured by the percent of LDH (lactate dehydrogenase) released into the medium. LDH activity was determined by Cytotoxicity Detection Kit. The optical density of the colored product was measured at 492 and 690 nm by SpectraMax 340 Microplate reader (Molecular Devices). LDH released into the medium was measured by taking samples of the medium. Total LDH was determined by taking samples of cell extracts lysed by Triton X-100 at a final concentration of 1%.

### 6.2 Results

### 6.2.1 Induction of AR in HepG2 Cells

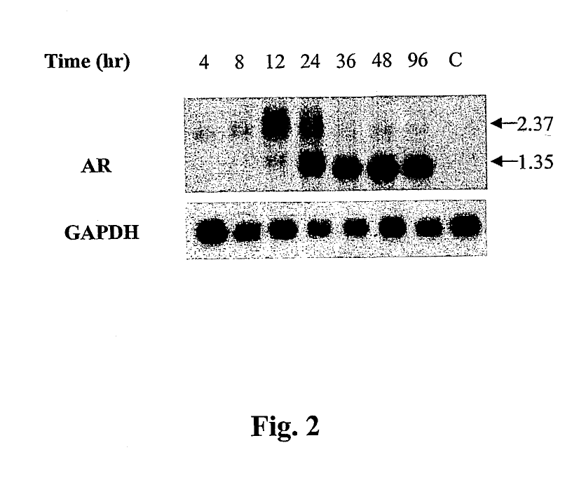

To simulate the overexpression of AR in liver cancers in cell culture, HepG2 cells were cultured in the hypertonic media containing 100 mM of NaCl. The hypertonic medium was shown to induce the expression of AR by HepG2 cells by about 15 fold compared to the isotonic medium (Nadkarni, V., Gabbay, K. H., Bohren, K. M., and Sheikh-Hamad, D., 1999, Osmotic response element enhancer activity. J. Biol Chem. 274:20185-90, which is hereby incorporated by reference in its entirety). As shown in FIG. 2, under the cell culture condition described above, hypertonic medium indeed induced the expression of AR.

### 6.2.2 Cytotoxicity of ND

Naphthalene is insoluble in water. Even when it was first dissolved in ethanol or DMSO, it quickly precipitates when added to the medium. Accordingly, ND, the metabolized product of naphthalene (see FIG. 1), rather than naphthalene, was used to test the cytotoxic activity of the drug that is activated AR overproduced by HepG2 cells.

As shown in FIG. 3, 500 μM of ND had no toxic effect on HepG2 cells cultured in isotonic medium. However, the same concentration of ND was highly toxic to HepG2 cells cultured in hypertonic medium as indicated by the large increase of LDH released into the medium after 5 hours of incubation with ND. The increased sensitivity of HepG2 cells cultured in hypertonic medium towards ND is due to the overexpression of AR. This is supported by the fact that AL1576, an inhibitor of AR (ARI), can protect these cells against the toxic effects of ND. At 50 μM, ND has no effect on HepG2 cells cultured in either isotonic or hypertonic medium.

### 6.3 Conclusions

The concept of taking advantage of the activities of the enzymes that are overexpressed in liver cancers to convert non-toxic chemicals to cytotoxic drugs was demonstrated in cell culture. ND was used to show that AR can convert this relatively non-toxic chemical to a highly cytotoxic drug. HepG2 cells were 10 times more sensitive to ND when cultured in hypertonic medium than when they were cultured in isotonic medium. The fact that AR inhibitor can protect the hypertonic HepG2 cells against the toxic effect of ND indicates that the increased sensitivity towards this chemical is the result of overexpression of AR that converts ND to a highly cytotoxic NQ. These methods based on the same concept have a great utility in designing and/or screening for new drugs to treat HCC as well as other types of cancers that overexpress specific enzymes capable of converting non-cytotoxic compounds to highly cytotoxic compounds.

ND is a potential drug candidate to treat HCC. Although ND is a metabolic product of naphthalene which causes cataract in animal models, appropriate amounts of this drug can be determined that would kill HCC specifically without causing cataract or damages to other tissues. Alternatively, ND can be used as part of the chemoembolization therapy.

Although naphthalene was not tested in the cell culture system described above because of the solubility problem, it may be a better candidate drug to treat HCC because its conversion to ND occurs only in the liver. This can be tested in animal models where naphthalene can be administered orally with oil as a solvent.

The disclosures of various publications referenced in this application are hereby incorporated by reference in their entireties in order to more fully describe the relevant art to which this invention pertains.

Those skilled in the art will recognize, or be able to ascertain many equivalents to the specific embodiments of the invention described herein using no more than routine experimentation. Such equivalents are intended to be encompassed by the following claims.

What is claimed is:

1. A method of screening for an anti-cancer drug comprising:
contacting a cancer cell overexpressing an enzyme with a candidate drug in the presence or absence of an inhibitor to the enzyme;
measuring a level of a substance correlatable with the level of cell death as a result of exposure of the cell to the drug; and
comparing the levels of the substance in the presence and absence of the inhibitor;
wherein a difference in the level of the substance in the absence of the inhibitor indicates that the drug has an anti-cancer activity against the cancer cell overexpressing the enzyme.

2. The method according to claim 1, wherein the cancer cell is liver cancer.

3. The method according to claim 2, wherein the cancer cell is hepatocellular carcinoma cell.

4. The method according to claim 2 or 3, wherein the enzyme is aldose reductase or ARL-1 or both.

5. The method according to claim 4, wherein the inhibitor is AL1567.

6. The method according to claim 1, 2, or 3, wherein the substance to be measured is LDH.

7. A method of treating or ameliorating cancer comprising administering to a subject in need of a treatment a therapeutically effective amount of the drug having an anti-cancer activity as determined by the method according to claim 1.

8. The method according to claim 7, wherein cancer is liver cancer.

9. The method according to claim 8, wherein cancer is hepatocellular carcinoma.

10. The method according to claim 7, 8 or 9, wherein the drug is administered orally.

11. The method according to claim 7, 8 or 9, wherein the drug is administered parenterally.

12. The method according to claim 11, wherein the drug is administered intravenously, subcutaneously, or intramuscularly.

13. The method according to claim 8 or 9, wherein the drug is administered intra-hepatic-arterially.

14. The method according to claim 8 or 9, the drug is administered by chemoembolization.

15. A method of treating or ameliorating liver cancer comprising administering to a subject in need of a treatment a therapeutically effective amount of naphthalene or a derivative thereof.

16. The method according to claim 15, wherein the liver cancer is hepatocellular carcinoma.

17. The method according to claim 15 or 16, wherein naphthalene or a derivative thereof is administered orally.

18. The method according to claim 17, wherein the derivative of naphthalene is naphthalene-1,2-dihydrodiol.

19. The method according to claim 15 or 16, wherein naphthalene or a derivative thereof is administered parenterally.

20. The method according to claim 19, wherein naphthalene or a derivative thereof is administered intravenously, subcutaneously, or intramuscularly.

21. The method according to claim 19, the derivative of naphthalene is naphthalene-1,2-dihydrodiol.

22. The method according to claim 15 or 16, wherein naphthalene or a derivative thereof is administered intra-hepatic-arterially.

23. The method according to claim 15 or 16, wherein naphthalene or a derivative thereof is administered by chemoembolization.

24. A pharmaceutical composition for treating or ameliorating cancer comprising a therapeutically effective amount of a drug having an anti-cancer activity as determined by the method according to claim 1 and a pharmaceutically acceptable carrier.

25. The composition according to claim 24, wherein cancer is liver cancer.

26. The composition according to claim 25, wherein the liver cancer is hepatocellular carcinoma.

27. A pharmaceutical composition for treating or ameliorating liver cancer comprising a therapeutically effective amount of naphthalene or a derivative thereof and a pharmaceutically acceptable carrier.

28. The composition according to claim 27, wherein the liver cancer is hepatocellular carcinoma.

29. The composition according to claim 27 or 28, wherein the derivative of naphthalene is naphthalene-1,2-dihydrodiol.

30. The composition according to claim 24, 25, 26, 27 or 28, wherein the composition is in a solid form for oral administration.

31. The composition according to claim 30, wherein the solid form for oral administration is a tablet.

32. The composition according to claim 30, wherein the solid form for oral administration is a capsule.

33. The composition according to claim 24, 25, 26, 27 or 28, wherein the composition is in a liquid form.

34. The composition according to claim 24, 25, 26, 27 or 28, wherein the carrier is Lipiodol.

35. The composition according to claim 29, wherein the carrier is Lipiodol.

36. A kit comprising in one or more containers the pharmaceutical composition of claim 24, 25, 26, 27 or 28 and an instruction for use of the kit.

37. A kit comprising in one or more containers the pharmaceutical composition of claim 29 and an instruction for use of the kit.
